(19) United States
(12) Patent Application Publication
(10) Pub. No.: US 2026/0000366 A1
(43) Pub. Date: Jan. 1, 2026

(54) PORTABLE BABY CARRYCOT AND BABY STROLLER

(71) Applicant: WONDERLAND SWITZERLAND AG, Steinhausen (CH)

(72) Inventors: Manqun CHENG, Dongguan (CN); Guozhu ZENG, Dongguan (CN)

(21) Appl. No.: 19/251,229

(22) Filed: Jun. 26, 2025

(30) Foreign Application Priority Data
Jun. 27, 2024 (CN) 202410852242.7

(51) Int. Cl. A61B 5/00; A61B 5/11; A61B 5/113; B62B 7/14

(52) U.S. Cl. CPC A61B 5/746; A61B 5/1126; A61B 5/113; A61B 5/4809; A61B 5/6892; B62B 7/142; A61B 2503/04

(57) ABSTRACT

A portable baby carrycot includes: a carrycot body including a first side wall and a second side wall opposite to each other, and a support plate at a bottom, a lying area being above the support plate; a support assembly including a first support part on the first side wall and a second support part on the second side wall; a piezoelectric sensor arranged in the lying area to detect a respiratory movement of a baby; a signal processing circuit configured to receive and process the respiratory movement; a controller configured to generate a prompt message indicating a sleep state of the baby when the received and processed respiratory movement satisfies a preset condition; and an alert device configured to provide a caregiver with the prompt message indicating the sleep state of the baby.

### CROSS-REFERENCE TO RELATED APPLICATION

This application claims priority to Chinese Application No. 202410852242.7, titled “Portable Baby Carrycot and Baby Stroller” and filed on Jun. 27, 2024, which is incorporated herein by reference in its entirety.

### TECHNICAL FIELD

The present disclosure relates to the technical field of children products and, more specifically, to a portable baby carrycot and a baby stroller.

### BACKGROUND

A baby carrycot, also commonly known as a bassinet or a baby sleep sack, is a portable and safe sleep product specifically designed for newborns and infants, and its appearance is usually of a small, bounded box-like structure and is just right sized for a baby to lie flat, with stable sides at the periphery and a soft mattress at the bottom. The baby carrycot is usually provided with a handle or a baby stroller frame connector on its side, to make it easy to take along the baby. However, due to a relatively closed mechanism of the carrycot, it is not easy to view the condition of the baby inside the carrycot from the outside, which is not convenience for a caregiver to take care of the baby.

### SUMMARY

Embodiments of the present disclosure provide a portable baby carrycot, including: a carrycot body including a first side wall and a second side wall opposite to each other, and a support plate at a bottom, a lying area being above the support plate; a support assembly configured to support the carrycot body, the support assembly including a first support part on the first side wall and a second support part on the second side wall; a piezoelectric sensor arranged in the lying area to detect a respiratory movement of a baby; a signal processing circuit configured to receive and process the respiratory movement detected; a controller configured to generate a prompt message indicating a sleep state of the baby when the received and processed respiratory movement satisfies a preset condition; and an alert device configured to provide a caregiver with the prompt message indicating the sleep state of the baby. A length of the piezoelectric sensor is greater than a width of the piezoelectric sensor, and a direction of a connection line between the first support part and the second support part is orthogonal to a direction of the length of the piezoelectric sensor.

Embodiments of the present disclosure provide a baby stroller, including: a frame including a first side and a second side opposite to each other, the first side and the second side having a first groove and a second groove, respectively; and a baby carrycot. The baby carrycot includes: a carrycot body including a first side wall and a second side wall opposite to each other, and a support plate at a bottom, a lying area being above the support plate; a support assembly configured to support the carrycot body, the support assembly including a connection assembly for connection with the frame, the connection assembly including a first connection assembly on the first side wall and a second connection assembly on the second side wall, and the first connection assembly and the second connection assembly being configured to engage with the first groove and the second groove, respectively; a piezoelectric sensor arranged in the lying area to detect a respiratory movement of a baby; a signal processing circuit configured to receive and process the respiratory movement detected; a controller configured to generate a prompt message indicating a sleep state of the baby when the received and processed respiratory movement satisfies a preset condition; and an alert device configured to provide a caregiver with the prompt message indicating the sleep state of the baby. A length of the piezoelectric sensor is greater than a width of the piezoelectric sensor, and a direction of a connection line between the first connection assembly and the second connection assembly is orthogonal to the direction of the length of the piezoelectric sensor.

It should be understood that the above general description and the subsequent detailed description are only exemplary and explanatory, and shall not limit the present disclosure.

### BRIEF DESCRIPTION OF THE DRAWINGS

FIG. 1 is a perspective view of a baby stroller according to an exemplary embodiment.

FIG. 2 is a side view of the baby stroller shown in FIG. 1.

FIG. 3 is a side view of a portable baby carrycot according to an exemplary embodiment.

FIG. 4 is a top view of the portable baby carrycot shown in FIG. 3.

FIG. 5 is a perspective view of the portable baby carrycot shown in FIG. 3.

FIG. 6 is a side view of a portable baby carrycot according to another exemplary embodiment.

FIG. 7 is a side view of a portable baby carrycot according to still another exemplary embodiment.

FIG. 8 is a schematic view of a sleeping pad and a piezoelectric sensor according to an exemplary embodiment.

FIG. 9 is a block diagram of a portable baby carrycot or a baby stroller according to an exemplary embodiment.

FIG. 10 is a schematic view illustrating generation of uniform signals when a baby is breathing normally, according to an exemplary embodiment.

FIG. 11 is a top view of a portable baby carrycot according to another exemplary embodiment.

FIG. 12 is a top view of a portable baby carrycot according to still another exemplary embodiment.

FIG. 13 is a top view of a portable baby carrycot according to yet another exemplary embodiment.

FIG. 14 is a flowchart of a risk detection method according to an exemplary embodiment.

### REFERENCE NUMERALS

100—baby stroller; 1—frame; 14—wheel; 121a—first side; 121b—second side; 111a—first groove; 111b—second groove; X—longitudinal direction; Y—transverse direction;
2—baby carrycot; 21—carrycot body; 251—first side wall; 252—second side wall; 254—front side wall; 255—rear side wall; 253—bottom; 24—support plate; 242—lying area; 3—sleeping pad;
23—support component; 23a—first support part; 23b—second support part; 23ab—direction of connection line between first support part 23a and second support part 23b; 241—through-hole; 39—centerline;
31—piezoelectric sensor; L—length of the piezoelectric sensor 31; W—width of the piezoelectric sensor 31;
32—signal processing circuit; 33—controller; 27—alert device;
34—communication module; 35—Bluetooth module; 36—cloud server; 37—mobile phone; 38—uniform signal;
5—support assembly; 22—connection assembly; 22a—first connection assembly; 22b—second connection assembly;
22ab—direction of connection line between first connection assembly 22a and second connection assembly 22b;
214—handle; 214a—first end of handle 214; 214b—second end of handle 214; 214ab—length direction of handle;
211—upper frame support tube; 212—carrycot support tube; 25—cloth cover; 26—sunshade;
4—baby; 41—head; 42—chest; 43—feet; 900—sleep detection device.

### DETAILED DESCRIPTION

Exemplary embodiments of the present disclosure will be exemplified with reference to the accompanying drawings. It can be understood that structures of the portable baby carrycot and the baby stroller according to embodiments of the present disclosure are not limited to the following illustration.

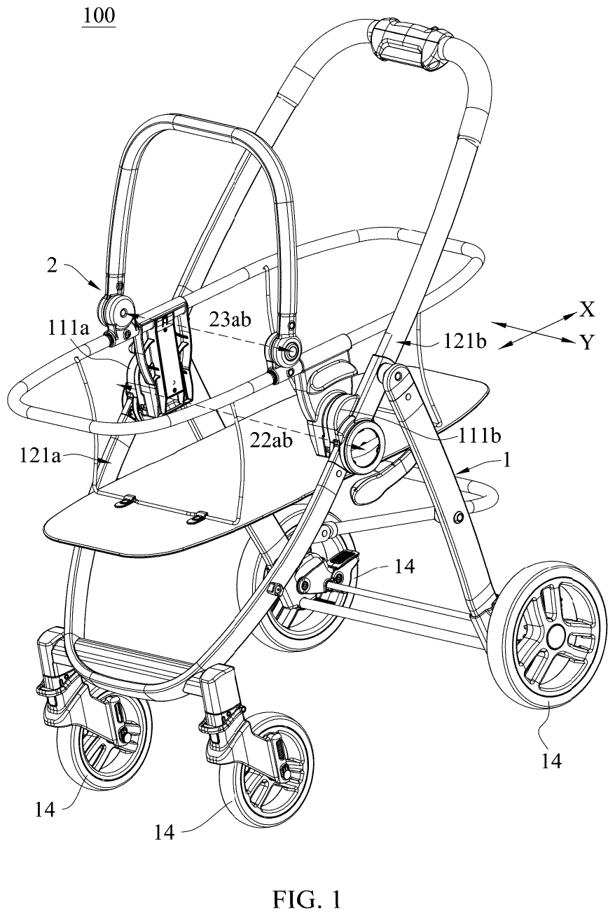

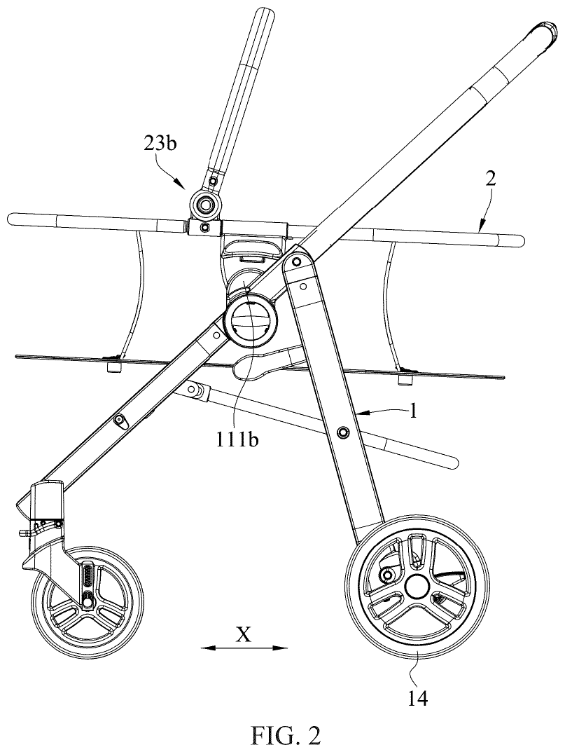

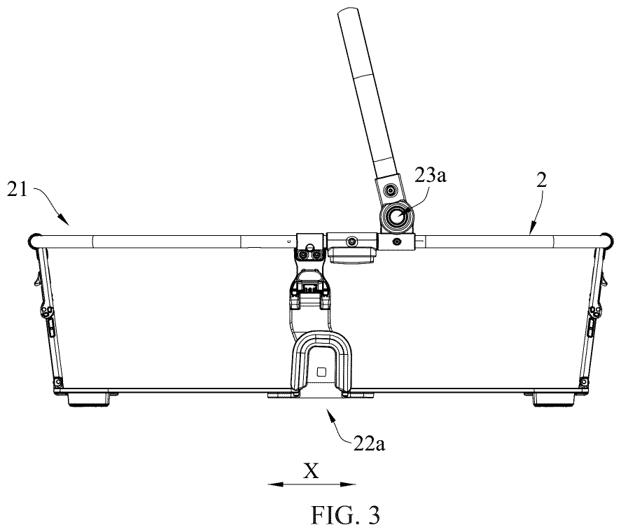

For example, as shown in FIGS. 1 to 12, a baby stroller 100 includes a frame 1 and a baby carrycot 2 on the frame 1. The frame 1 includes a first side (which may be a left side or a right side of the frame 1) 121a and a second side (which may be the right side or the left side of the frame 1) 121b. A bottom end of the frame 1 is provided with two rows of wheels 14, and a middle part of the frame 1 is provided with a first groove 111b on the first side 121a of the frame 1 and a second groove 111b on the second side 121b of the frame 1, both of which are configured to snap-fit with the baby carrycot 2. The baby carrycot 2 includes a carrycot body 21, a support assembly 5, a piezoelectric sensor 31, a signal processing circuit 32, a controller 33, and an alert device 27. The first side 121a of the frame 1 is located on a same side as a first side wall 251 of the baby carrycot 2, and the second side 121b of the frame 1 is located on a same side as a second side wall 252 of the baby carrycot 2.

The carrycot body 21 includes the first side wall 251 and the second side wall 252 opposite to each other, as well as a support plate 24 at a bottom 253. Above the support plate 24, there is a lying area 242.

The support assembly 5 is used to support the carrycot body 21. The support assembly 5 may include a connection assembly 22 for connection with the frame 1. The connection assembly 22 includes a first connection assembly 22a on the first side wall 251 and a second connection assembly 22b on the second side wall 252. The first connection assembly 22a and the second connection assembly 22b are used to engage with the first groove 111a and the second groove 111b, respectively, so that the baby carrycot 2 may be detachably mounted on or removed from the frame 1. The baby carrycot 2 according to embodiments of the present disclosure may be snap-fit with the frame 1 by two snap points (i.e., using the first groove 11a and the second groove 111b), which may prevent accidental unlocking and facilitate quick removal or installation of the baby carrycot 2 on the frame 1.

The piezoelectric sensor 31 is arranged in the lying area 242 to detect a respiratory movement of a baby 4 lying in the lying area 242. A length L of the piezoelectric sensor 31 is greater than a width W of the piezoelectric sensor 31. A direction 22ab of a connection line between the first connection assembly 22a and the second connection assembly 22b is orthogonal to a direction of the length L of the piezoelectric sensor 31. The baby stroller 100 engages with the first connection assembly 22a of the first side wall 251 and the second connection assembly 22b of the second side wall 252 of the baby carrycot 2 through the first groove 111a on the first side 121a and the second groove 111b on the second side 121b of the frame 1, so that the baby carrycot 2 is detachably connected to the frame 1.

The piezoelectric sensor 31 in the embodiments of the present disclosure operates based on the piezoelectric effect, and its sensitive element is made of a piezoelectric material. When subjected to a force, the piezoelectric material generates an electric charge on its surface, and the electric charge is converted into an electrical output proportional to the applied external force after amplification and impedance transformation by a charge amplifier and a measurement circuit. The piezoelectric sensor 31 is used to output a corresponding current signal/voltage signal according to the magnitude of pressure.

The signal processing circuit 32 is coupled to the piezoelectric sensor 31 and the controller 33, to receive, from the piezoelectric sensor 31, the respiratory movement of the baby 4 detected by the piezoelectric sensor 31, process the received respiratory movement of the baby 4, obtain the processed respiratory movement of the baby 4, and send the processed respiratory movement of the baby 4 to the controller 33. The controller 33 is coupled to the alert device 27 and is used to receive the processed respiratory movement of the baby 4 from the signal processing circuit 32 and determine whether the processed respiratory movement of the baby 4 satisfies a preset condition. If the processed respiratory movement of the baby 4 satisfies or conforms to the preset condition, the controller generates a prompt message indicating a sleep state of the baby 4 and sends the prompt message to the alert device 27. The alert device 27 is used to provide a caregiver of the baby 4 with the prompt message indicative of the sleep state of the baby 4 when the prompt message is received from the controller 33.

When the caregiver lies the baby 4 in the lying area 242 on the support plate 24 at the bottom 253 of the baby carrycot 2 and pushes the baby stroller 100, the baby stroller 100 will drive the baby carrycot 2 to sway along a longitudinal direction X of the carrycot body 21 (i.e., a front-rear direction when the caregiver pushes the baby stroller 100 forwards or backwards), and the swaying may cause noise or interference to the detection of the respiratory movement of the baby 4 lying in the lying area 242. The embodiments of the present disclosure provide the piezoelectric sensor 31 in the lying area 242, with the length L of the piezoelectric sensor 31 being greater than its width W, and the direction of the length of the piezoelectric sensor 31 being orthogonal to the direction 22ab of the connection line between the first connection assembly 22a and the second connection assembly 22b. Due to a significant difference between the noise caused by the swaying and the respiratory movement of the baby, the noise caused by the swaying may be subsequently distinguished and recognized by a processing procedure, and then be filtered out, thereby improving the accuracy of detecting respiration information of the baby. In addition, the piezoelectric sensor 31 in the embodiments of the present disclosure does not need to be in direct contact with the baby 4 in the lying area 242; for example, it may detect the respiration and sleep state of the baby 4, without need to be worn by the baby 4 on the wrist, which may improve the convenience of detection as well as the comfort of the baby 4, e.g., not causing interference to the baby 4.

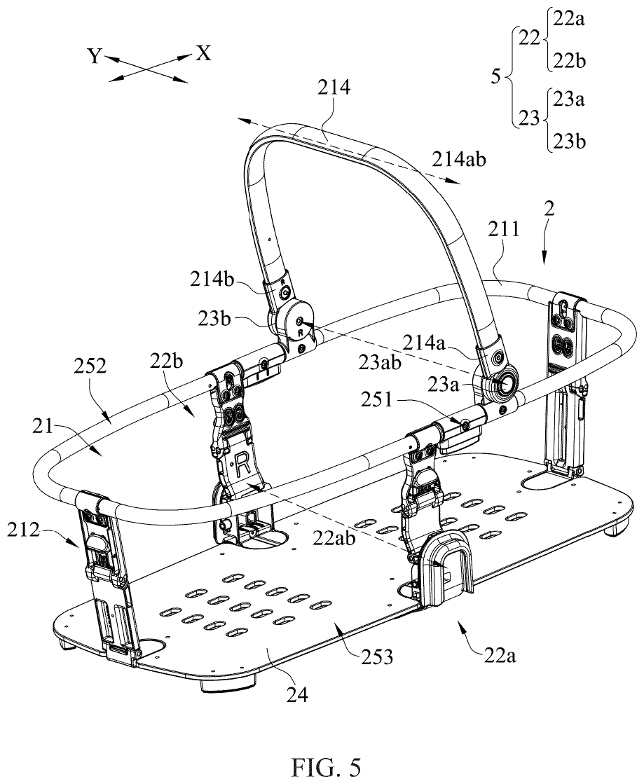

Referring to an embodiment shown in FIG. 5, the support assembly 5 may further include a first support part 23a on the first side wall 251 and a second support part 23b on the second side wall 252, in which the first support part 23a and the second support part 23b constitute a support component 23. A direction 23ab of a connection line between the first support part 23a and the second support part 23b is orthogonal to the direction of the length of the piezoelectric sensor 31.

Still referring to FIG. 5, the carrycot body 21 is provided with a handle 214 for carrying, and the handle 214 is arranged in a movable manner on the carrycot body 21. The caregiver may easily take along the baby carrycot 2 and the baby 4 inside the baby carrycot 2 by mounting the baby carrycot 2 on the frame 1 or by holding the handle 214 of the baby carrycot 2. Two ends of the handle 214 (referred to as a first end 214a and a second end 214b) are connected to the first support part 23a on the first side wall 251 and the second support part 23b on the second side wall 252, respectively. A length direction 214ab of the handle 214 is orthogonal to the direction of the length of the piezoelectric sensor 31.

The baby carrycot 2 in the embodiments of the present disclosure may be used independently by disengaging the first groove 111a and the second groove 111b of the baby stroller 100 from the first connection assembly 22a and the second connection assembly 22b of the baby carrycot 2, respectively. When the baby carrycot 2 is used alone, the caregiver may walk when lifting the handle 214. Since the two ends of the handle 214 are connected to the first support part 23a and the second support part 23b, respectively, the baby carrycot 2 sways back and forth during walk, causing the piezoelectric sensor 31 laid in the lying area 242 to simultaneously detect current/voltage changes induced by the noise caused by the swaying and the respiration of the baby 4 lying in the lying area 242. Due to the significant difference between the noise caused by the swaying and the respiratory movement of the baby, the noise caused by the swaying may be subsequently distinguished and recognized by the processing procedure, and then be filtered out, thereby improving the accuracy of detecting respiration information of the baby.

In the embodiments of the present disclosure, a length direction of the baby carrycot 2 is a long axis direction (also known as the longitudinal direction) X of the baby carrycot 2, and a width direction of the baby carrycot 2 is a short axis direction (also known as a transverse direction) Y. A length of the baby carrycot 2 is greater than a width of the baby carrycot 2. The X-axis direction intersects with or is orthogonal to the Y-axis direction.

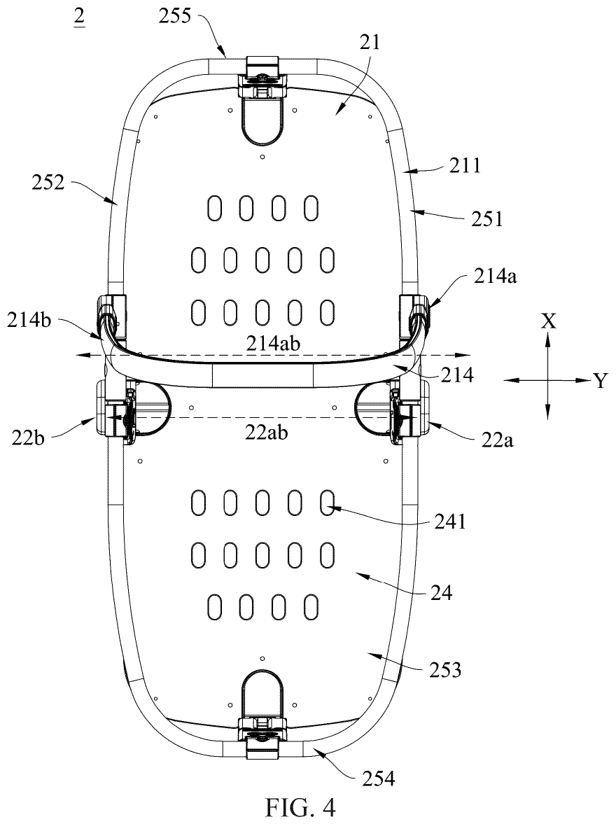

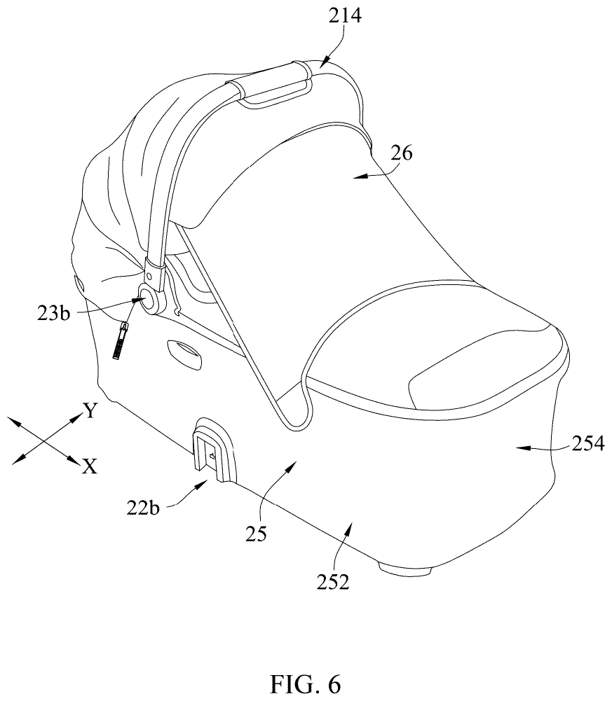

Referring to FIGS. 4 and 5, the carrycot body 21 also includes an upper frame support tube 211, and a carrycot support tube 212 connected to both the upper frame support tube 211 and the support plate 24. The carrycot support tube 212 may be folded or unfolded relative to the upper frame support tube 211. The baby carrycot 2 collapses by folding the carrycot support tube 212, and is fixed to the support plate 24 when the carrycot support tube 212 is unfolded. Therefore, the upper frame support tube 211, the carrycot support tube 212, and the support plate 24 constitute the frame of the entire baby carrycot 2, and then a cloth cover (as shown in FIG. 6) is attached to form the baby carrycot 2 capable of carrying the baby 4.

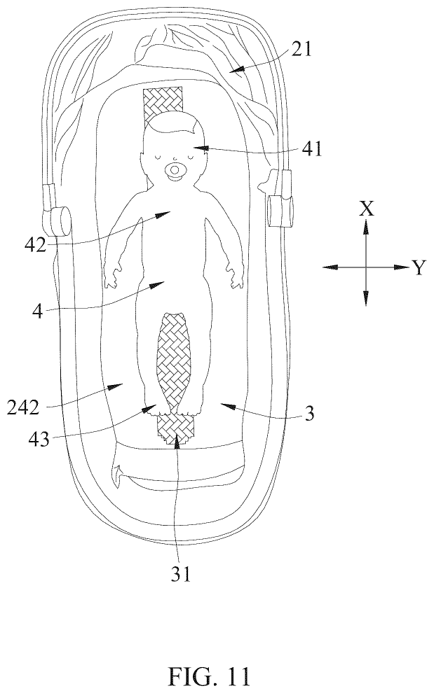

In some embodiments, a pair of upper frame support tubes 211 extending along the length direction of the baby carrycot 2, the connection assembly 22, and the cloth cover 25 attached to the upper frame support tubes 211 constitute the first side wall 251 and the second side wall 252 of the baby carrycot 2. When the baby 4 is lying in the baby carrycot 2, the first side wall 251 (also known as a left side wall) is adjacent to a left arm of the baby 4, and the second side wall 252 (also known as a right side wall) is adjacent to a right arm of the baby 4, as shown in FIG. 11. In other embodiments, the first side wall 251 and the second side wall 252 may also be areas constituted by the upper frame support tubes 211 extending along the width direction of the baby carrycot 2, the connection assembly 22, and the cloth cover 25. In yet other embodiments, the first side wall 251 and the second side wall 252 may also be areas constituted by the upper frame support tubes 211 extending along the length direction or the width direction of the baby carrycot 2 and the connection assembly 22, i.e., the cloth cover 25 may be excluded, for example, as shown in FIG. 5.

In some optional embodiments, the support plate 24 and the connection assembly 22 are made of hard materials such as polypropylene (PP), and the upper frame support tube 211 is made of metal materials. An inner side of the frame of the carrycot body 21 is also provided with a cushioning member (not shown in the figures) formed by foaming of a foam material, and the cushioning member covers side walls between the frame support tube 211 and the support plate 24, as well as an upper surface of the support plate 24, making the structure of the baby carrycot 2 stable and lightweight. The foam material may be one of or a combination of more of polyvinyl chloride (PVC), polypropylene foam plastic, expandable polypropylene (EPP), expandable polystyrene (EPS), expandable polyolefin (EPO) or polyethylene (PE), as long as the foam material selected is lightweight, safe, and durable. The cushioning member may be considered as forming part of the first side wall 251, the second side wall 252, and the support plate 24.

Still referring to FIG. 6, the carrycot body 21 may also include a front side wall 254 (typically a side wall corresponding to the feet of the baby 4 when lying down) and a rear side wall 255 (typically a side wall corresponding to the head of the baby 4 when lying down). In an embodiment shown in FIG. 6, a sunshade 26 may also be added to the carrycot body 21 to shield the baby 4 from sunlight, wind, rain, or etc.

Referring to FIGS. 4 and 5, the support plate 24 is provided with one or more through-holes 241. In other embodiments, one or more side walls of the carrycot body 21 may be provided with one or more through-holes 241. The through-hole(s) 241 may be used for a shoulder strap and/or a waist strap to pass through, to secure the baby 4 inside the carrycot body 21, and may be also used for ventilation and air exchange inside the carrycot body 21, making the baby 4 more comfortable during use. At the same time, the arrangement of the through-hole(s) 241 also reduces processing materials of the carrycot body 21, and may further reduce the weight of the carrycot body 21, in order to further reduce the overall weight of the baby carrycot 2, and make the texture of the carrycot body 21 more lightweight.

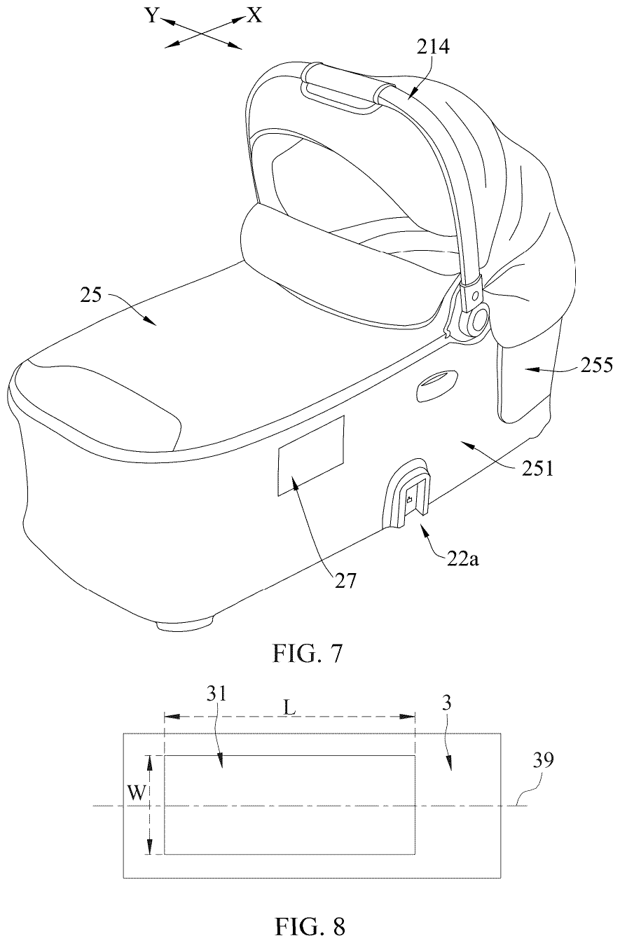

As shown in FIG. 7, the alert device 27 may be fixed on one side wall of the carrycot body 21, such as the second side wall 252, but the present disclosure is not limited thereto, as long as it is easy for the caregiver to observe an alert message displayed or broadcast by the alert device 27. For example, the alert device 27 may be fixed on the upper frame support tube 211.

In some embodiments, the baby carrycot 2 also includes a battery for powering a sleep detection device, or an external power interface. In other embodiments, the external power interface of the baby carrycot 2 is arranged at the connection assembly 22; the frame 1 is provided with a battery, and a power supply interface is provided at the first groove 111a or the second groove 111b of the frame 1; when the baby carrycot 2 is mounted on the frame 1, the external power interface at the connection assembly 22 is coupled with the power supply interface at the first groove 111a or the second groove 111b, thereby supplying power to the sleep detection device in the baby carrycot 2. Thus, the weight of the baby carrycot 2 may be reduced, and detection of the sleep state of the baby 4 automatically starts when the baby carrycot 2 is mounted to the frame 1, to prevent the caregiver from failing to pay timely attention to the condition of the baby 4 during travelling.

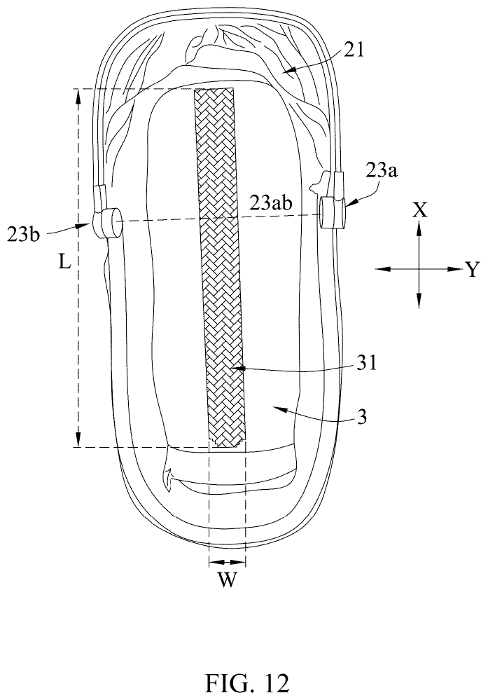

As shown in FIGS. 11 and 12, in an exemplary embodiment, a sleeping pad 3 is laid on the support plate 24, and the sleeping pad 3 is made of a soft sheet made of a foam material or a pad filled with down or other materials inside a cloth cover. The piezoelectric sensor 31 is arranged within or on an upper surface of the sleeping pad 3. By laying the sleeping pad 3 on the support plate 24 and arranging the piezoelectric sensor 31 within or on the sleeping pad 3, the sleeping pad 3 locates in the lying area 242 for the baby 4, which may improve the comfort of the baby 4 and reduce the disturbance caused by the swaying of the support plate 24.

In an exemplary embodiment, the piezoelectric sensor 31 is a one-piece thin film piezoelectric sensor. The direction of the length of the piezoelectric sensor 31 is parallel to the longitudinal direction X of the baby carrycot 2, and is orthogonal to the length direction of the handle 214, the direction 22ab of the connection line between the first connection assembly 22a and the second connection assembly 22b, and the direction 23ab of a connection line between the first support part 23a and the second support part 23b. The embodiment of the present disclosure only requires the provision of the one-piece piezoelectric sensor 31, which may have a simpler manufacturing process, lower cost, and easier operation when laid in the lying area 242.

In an exemplary embodiment, a centerline of the piezoelectric sensor 31 along its length direction coincides with a centerline of the lying area 242 along its longitudinal direction X. In some embodiments, the piezoelectric sensor 31 is located in the middle of the lying area 242 along its length direction. Thus, when the baby 4 lies in the lying area 242, since the lying area 242 is usually slightly larger than the body width of the baby 4, such arrangement may ensure that the baby 4 may lie on the piezoelectric sensor 31, thereby improving the accuracy of detection.

For example, as shown in FIG. 8, the piezoelectric sensor 31 is arranged on the sleeping pad 3. The centerline of the piezoelectric sensor 31 along its length direction coincides with the centerline of lying area 242 along its longitudinal direction, as shown by a centerline 39.

In an exemplary embodiment, the length of the piezoelectric sensor 31 is equal to a length of the lying area 242. In some embodiments, the piezoelectric sensor 31 may extend from head to tail along the length of the lying area 242 inside the carrycot body 21, which on the one hand makes it easy to fix the piezoelectric sensor 31 on top of the lying area 242, and on the other hand may detect swaying of the head 41, chest 42, and feet 43 of the baby 4, as well as the respiration of the baby, thereby further improving the accuracy of detection.

For example, as shown in FIG. 12, the sleeping pad 3 is laid on the bottom of the carrycot body 21, and a length of the sleeping pad 3 is the length of the lying area 242. The one-piece piezoelectric sensor 31 is provided in the center of sleeping pad 3. The length of the piezoelectric sensor 31 is equal to or almost equal to the length of sleeping pad 3. It can be understood that the term “equal” in the embodiments of the present disclosure allows for an appropriate deviation range, as long as it does not affect the ability of detecting the head 41, chest 42, feet 43 and respiratory movement of the baby when the baby 4 is lying on the piezoelectric sensor 31.

In an embodiment shown in FIG. 12, the piezoelectric sensor 31 may have a size of 835 mm (length)*60 mm (width) for example, but the present disclosure is not limited thereto. The length and width of the piezoelectric sensor 31 may be adjusted according to sizes of the length and width of the lying area 242. When the length of the lying area 242 increases, the length of the piezoelectric sensor 31 may also increase accordingly; when the width of the lying area 242 increases, the width of the piezoelectric sensor 31 may also increases accordingly. The width of the piezoelectric sensor 31 may be smaller than the width of lying area 242.

In an exemplary embodiment, the length of the piezoelectric sensor 31 is smaller than the length of the lying area 242, and at least part of the head 41 and chest 42 of the baby 4 lying in the lying area 242 is located on the piezoelectric sensor 31, i.e., at least the respiration of the baby 4 may be detected.

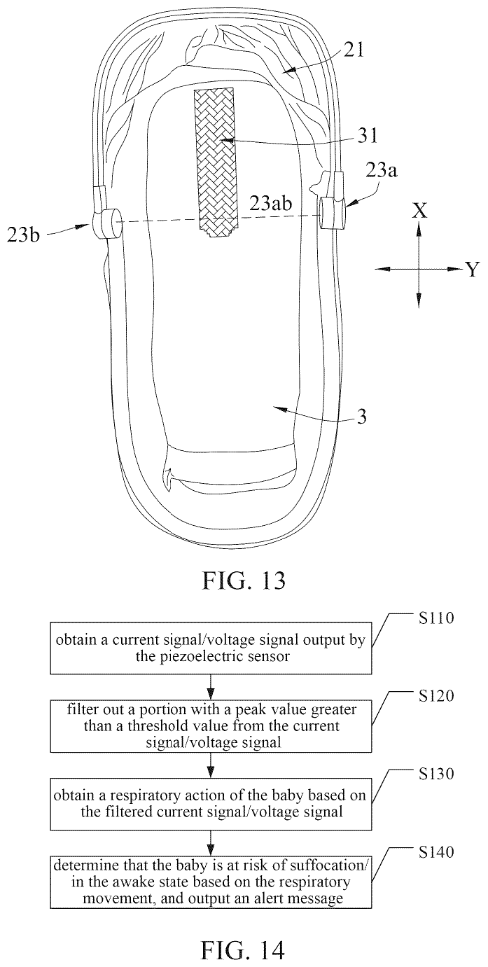

For example, as shown in FIG. 13, the length of the piezoelectric sensor 31 is approximately half or more than half or less than half of the length of the lying area 242. For example, the piezoelectric sensor 31 extends from the direction 23ab of a connection line between the first support part 23a and the second support part 23b to the rear side wall 255 of the baby carrycot 2, i.e., covering only a position corresponding to the upper half of the body of the baby 4. As long as at least a part of the head 41 and at least a part of the chest 42 of the baby 4 may be located on the piezoelectric sensor 31 when the baby 4 is lying in the lying area 242, the respiratory movement of the baby may be accurately detected, which may further reduce the cost of the piezoelectric sensor.

In an exemplary embodiment, the piezoelectric sensor 31 adopts a thin film piezoelectric sensor/piezoelectric thin film sensor. The piezoelectric thin film sensor is very thin, lightweight, and very soft. The application of the thin film piezoelectric sensor in the embodiments of the present disclosure may improve the comfort of the baby and allow the baby to barely feel the presence of the piezoelectric thin film sensor. In an exemplary embodiment, the thin film piezoelectric sensor is made of PVDF (polyvinylidene difluoride), but the present disclosure is not limited thereto. The thin film piezoelectric sensor made of PVDF is very sensitive to small changes in force and has high sensitivity, and is capable of operating over a wide frequency range and may be used to measure signals and fluctuations at different frequencies.

In an exemplary embodiment, the piezoelectric sensor 31 outputs a current signal/voltage signal of corresponding intensity based on the magnitude of pressure exerted on the piezoelectric sensor 31 by the body of the baby 4. When the baby 4 is breathing, the heaving of the chest 42 will produce weak pressure fluctuations, which may be captured and converted into an electrical signal by the piezoelectric sensor 31. From the electrical signal, the frequency change of the baby's respiration may be extracted to determine the sleep status/state of the baby (e.g., whether the baby is in an awake state or in a sleeping state, or whether there is a risk of suffocation, etc.), and to prompt the caregiver. In addition, in some embodiments, the heart rate of the baby may also be detected by the electrical signal of the piezoelectric sensor 31, and the principle is basically the same as detection of the respiratory rate, which will not be repeated herein.

When the caregiver pushes the baby stroller 100 outdoors, the wheels 14 move vertically due to an uneven ground surface, causing the baby carrycot 2 to sway forwards or backwards around an axis of the connection line between the first connection assembly 22a and the second connection assembly 22b. In addition, when the caregiver does not use the frame 1, but lifts the handle 214 of the baby carrycot 2 and walks, there will also be swaying with the length direction of the handle 214 as an axis. The amplitude of swaying is related to the evenness of the ground surface and stride frequency of the caregiver. During the swaying, the body of the baby 4 exerts irregularly varying interference pressure on the piezoelectric sensor 31, which is superimposed on the pressure generated by respiration of the baby, and consequently will cause noise or interference to the detection of the respiration information of the baby lying in the lying area, affecting the accuracy of detecting the sleep state.

The direction of the length of the piezoelectric sensor 31 is orthogonal to the direction of the axis of the swaying as describe above, and covers the head 41, chest 42, and/or feet 43 of the baby. When the carrycot body 21 sways back and forth, the head and feet of the baby 4 are farther away from the axis of the swaying, so the amplitude of the swaying of the head and feet is relatively large. The piezoelectric sensor 31 detects that the interference pressure generated by the swaying of the head and feet of the baby 4 will be significantly greater than the weak pressure generated by the respiration of the baby 4. Thus, an effective signal generated by respiration and a noise signal generated by swaying may be quickly and conveniently distinguished using the signal processing circuit 32.

In the embodiments of the present disclosure, the sleep detection device is arranged inside the baby carrycot 2, and includes the piezoelectric sensor 31 located above the lying area 242 of the baby. When the baby 4 lies in the lying area 242, the sleep state of the baby 4 in the baby carrycot 2 may be detected to improve the safety of the baby 4 in the baby carrycot 2 and make it convenient for the caregiver to timely take care of the baby 4 in the baby carrycot 2.

In an exemplary embodiment, the portable baby carrycot 2 also includes an alert control circuit. The alert control circuit is used to receive and process the current signal/voltage signal output by the piezoelectric sensor 31, obtain the respiratory movement of the baby, and output an alert message when it is determined based on the respiratory movement that the baby is at risk of suffocation/in the awake state (i.e., satisfying the preset condition).

In an exemplary embodiment, the alert control circuit includes a signal processing circuit configured to receive and filter out a portion, in which a peak value is greater than a threshold value, of the respiratory movement detected by the piezoelectric sensor; a controller configured to receive the respiratory movement after being processed by the signal processing circuit, obtain the respiratory information of the baby based on the respiratory movement after being filtered, determine whether or not the baby is at risk of suffocation/in the awake state based on the respiratory information, and output an alert message when it is determined that the baby is at risk of suffocation/in the awake state. The alert message may be used to indicate that the baby is at risk of suffocation/in the awake state.

In the embodiments of the present disclosure, due to light respiration of the baby during sleep, the peak of the current signal/voltage signal caused by the swaying of the carrycot body and output by the piezoelectric sensor is greater than the peak of the current signal/voltage signal caused by the baby's respiration during sleep, so that the signal processing circuit may first filter out a portion with a peak value greater than a threshold value from the current signal/voltage signal, and then the controller may use the filtered current signal/voltage signal to detect the respiratory movement, thereby removing the noise caused by swaying, improving the accuracy of detecting the respiratory movement of the baby, and enhancing the safety of the baby when the respiratory movement is used to determine whether the baby is at risk of suffocation/in the awake state.

In an exemplary embodiment, the alert control circuit further includes an alert device configured to display or broadcast an alert message, and the alert device is mounted on a side wall of the carrycot body or on the handle.

In an exemplary embodiment, the alert device may include any one or more of a display device (such as a display screen), a buzzer device, or an electronic device (such as a mobile phone). The alert device may be arranged in a position on the carrycot for easy observation, such as on any one or more side walls of the carrycot, or at the front or rear of the carrycot. Specifically, the alert device may be fixed on the upper frame support tube of the carrycot, for example. However, the present disclosure is not limited thereto, and the alert device may also be arranged in other positions of the carrycot, such as in an appropriate position on the handlebar or the handle. Optionally, the alert device may also include a switch button or key to allow the caregiver to turn on or off the alert device.

For example, by installing inside the carrycot the sleep detection device including the above-mentioned piezoelectric sensor, the sleep detection device may be used to collect respiration information (and optionally heart rate and/or movement information) of an infant/a child to determine whether the infant/child is suffocating and whether the infant/child is awake, and by prompting the state of the infant/child through the alert device, the caregiver may take care of the infant/child inside the carrycot in a timely manner, making the infant/child more comfortable and safe. That is, the embodiments of the present disclosure provide a baby carrycot with a sleep monitoring function, including a box body and a sleep detection device inside the box body; the sleep detection device includes a piezoelectric sensor located in a lying area of a baby, i.e., below the baby's body, and is configured to collect baby information, such as respiration information of the baby. The piezoelectric sensor may be electrically connected (directly or indirectly, in a wireless or wired manner) to an alert control circuit/an alert control device. The alert control device includes a signal processing circuit, a controller (also known as a “main controller” or “processor” or “main control processor”), and an alert device. A current signal/voltage signal collected by the piezoelectric sensor is processed by the signal processing circuit, judged by the main controller, and output by the alert device. The output information may include a sleep status of the baby, mainly including whether the baby is awake and whether the baby is suffocating. When the baby has woken up from sleep or there is a risk of suffocation, an alert message will be issued, displayed on a display screen mounted on the carrycot or broadcast by a buzzer on the carrycot.

In an exemplary embodiment, the alert control circuit further includes a communication assembly. The communication assembly is configured to output the alert message to the electronic device for display or broadcast.

The main control processor processes, analyzes, and calculates data collected from the piezoelectric sensor through the signal processing circuit to obtain at least one of respiratory information (e.g., respiratory rate), heart rate, and movement state/information of the baby, and converts it into the baby's sleep quality/sleep status through algorithm analysis. When the baby wakes up or is at risk of suffocation, the alert message is issued, and the alert message is pushed to the electronic device through Bluetooth or wireless network, so that the caregiver may know at the first time that the baby has woken up or is at risk of suffocation, and take timely care.

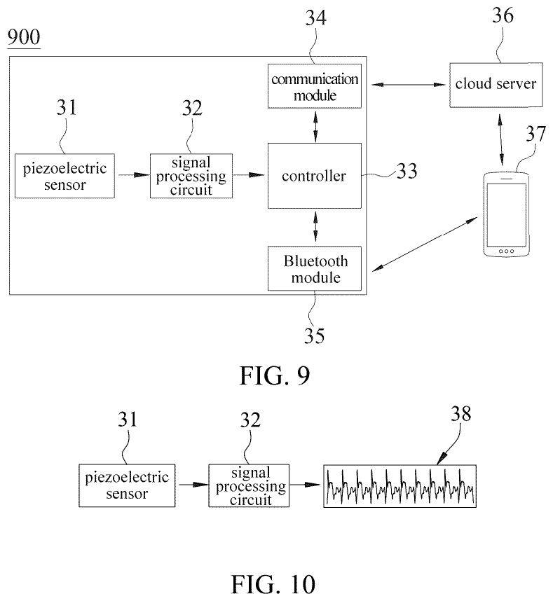

For example, as shown in FIG. 9, a sleep detection device 900 includes a piezoelectric sensor 31 and an alert control circuit. The alert control circuit may include a signal processing circuit 32 and a controller 33. The piezoelectric sensor 31 is electrically connected to the signal processing circuit 32 to receive a current signal/voltage signal collected by the piezoelectric sensor 31, and filter the current signal/voltage signal to remove a portion with a peak value greater than a threshold value. The signal processing circuit 32 is also electrically connected to the controller 33, and transmits to the controller 33 the current signal/voltage signal from which the portion with the peak value greater than the threshold value has been removed. The threshold value may be set according to actual needs.

The controller 33 processes the received current signal/voltage signal from which the portion with the peak value greater than the threshold value has been removed, extracts the baby's respiration information, and determines whether the baby is at risk of suffocation/in an awake state. If there is a risk of suffocation/in the awake state, an alert message is generated. Optionally, the sleep detection device 900 may also include a communication module 34 and/or a Bluetooth module 35, the communication module 34 and/or the Bluetooth module 35 being electrically connected to the controller 33. The above communication assembly may include the communication module 34 and/or the Bluetooth module 35. The communication module 34 may use a wireless network, such as 3G/4G/5G technology, to transmit the alert message generated by the controller 33 to a cloud server 36. The cloud server 36 communicates wirelessly with an electronic device such as a mobile phone 37, and sends the alert message to the mobile phone 37 for display or broadcast. Alternatively, the main controller 33 may directly send the alert message to the mobile phone 37 for display or broadcast through the Bluetooth module 35.

It can be understood that the signal processing circuit 32 and the controller 33 may be mounted on the baby carrycot 2, or may be mounted on the baby stroller 100, or may be installed on the cloud server 36, or may be installed in the electronic device such as the mobile phone 37.

In the embodiments of the present disclosure, the baby's respiration may cause the piezoelectric sensor to generate a weak signal change. After signal amplification, unwanted signals (such as noise caused by swaying) are filtered out, and useful signals are counted to calculate the baby's respiratory rate, heart rate, and movement status. When the baby performs movements (such as turning over), since the movements are larger than respiration, the signals are stronger and irregular, and it may be considered that the baby has woken up; the fewer movements the baby performs, the more even the respiration is, and the better the sleep quality is considered to be.

During even respiration, if the baby suddenly performs continuous large movements, it is considered that the baby has woken up; during even respiration, if the signal suddenly diminishes or stops, it is considered that the baby is at risk of suffocation.

The controller 33 may adopt a microcontroller unit (MCU), a digital signal processing (DSP) controller, a central processing unit (CPU), or etc.

The signal processing circuit 32 may include an amplifier, a comparator, and a filter. An output terminal of the piezoelectric sensor 31 is connected to a forward input terminal and a reverse input terminal of the amplifier. The amplifier amplifies the current signal/voltage signal output by piezoelectric sensor 31. An input terminal of the comparator is connected to an output terminal of the amplifier, and an output terminal of the comparator is connected to an input terminal of the filter. The comparator is used to compare each peak of the amplified current signal/voltage signal with a preset threshold. When the peak of the amplified current signal/voltage signal is greater than or equal to the preset threshold, it is filtered out by the filter. The filter is connected to the controller 33.

It can be understood that in order to save power consumption and improve battery endurance, when the infant or child leaves the lying area, a signal generated by the piezoelectric sensor 31 will be smaller than a preset signal threshold, in which case another comparator may be provided. If the other comparator determines that the signal generated by the piezoelectric sensor 31 is smaller than the signal threshold through comparison, the main controller 33 may control the sleep detection device 900 to automatically enter a sleep mode, reducing the power consumption of the sleep detection device, so that the product may be used for a longer period of time after a single charge, and is suitable for long-term monitoring of the sleep condition of the infant or child.

FIG. 10 shows a uniform signal 38 generated by the signal processing circuit 32 after processing the current signal/voltage signal collected by the piezoelectric sensor 31 when the baby is breathing normally in the carrycot.

FIG. 14 is a flowchart of a sleep status detection method using the carrycot of the aforementioned embodiments, according to an exemplary embodiment. As shown in FIG. 14, the method according to the embodiments of the present disclosure may include the following steps.

In S110, a current signal/voltage signal output by the piezoelectric sensor is obtained.

In S120, a portion with a peak value greater than a threshold value is filtered out from the current signal/voltage signal.

In S130, a respiratory movement of the baby is obtained based on the filtered current signal/voltage signal.

In the embodiments of the present disclosure, respiratory information may include any information related to respiration of the baby, such as one or more of respiratory rate (the number of breaths per unit of time), the number of breaths within a preset duration, respiratory spectrogram, etc. Here is an example of the respiratory rate.

In Step 140, based on the respiratory movement, it is determined that the baby is at risk of suffocation/in the awake state, and an alert message is output.

The frequency of noise caused by the swaying of the baby carrycot to the current signal/voltage signal output from the piezoelectric sensor is related to the evenness of the ground surface and is likely be the same as the baby's respiratory rate, but the peak of the current signal/voltage signal caused by the swaying is much higher than the frequency of the baby's respiration. Thus, the method according to the embodiments of the present disclosure first amplifies the current signal/voltage signal during signal processing, and then carries out filtration to filter out a signal with a larger peak; the rest is considered as a useful current signal/voltage signal caused by the baby's respiration. After filtering, spectral analysis is performed to process the current signal/voltage signal, so at to obtain the baby's respiratory rate. It is judged whether the respiratory rate is stable, decreasing or increasing, in order to determine whether the baby has woken up or is at risk of suffocation. Thus, the accuracy of detection and the safety of the baby may be enhanced.

Further, when obtaining the respiratory rate, for a characteristic in a respiratory signal of an infant or a child, which is prone to occurrence of a secondary wave interference signal, the method according to the embodiments of the present disclosure may filter out the secondary wave interference signal in the respiratory signal of the infant or the child, and then calculates the respiratory rate, thereby obtaining a more accurate respiratory rate.

When the human body is located next to the piezoelectric sensor, the piezoelectric sensor may sense a mechanical vibration pressure signal generated by human respiration, heartbeat, or body movement, and convert the mechanical vibration pressure signal into a current signal. When the human body is located next to the piezoelectric sensor, it means that the human body is on or close to the piezoelectric sensor to an extent that the piezoelectric sensor may detect a micro-motion signal of the human body.

In some embodiments, when the piezoelectric sensor obtains the current signal/voltage signal, it is transmitted to the signal processing circuit, and the signal processing circuit converts the current signal/voltage signal into an algorithm-processable digital signal through analog-to-digital conversion; the respiratory rate is obtained based on the current signal/voltage signal that has been converted to the digital signal.

In other embodiments, for the current signal/voltage signal after the signal with the larger peak is filtered out, the respiratory rate may be obtained by filtration using a bandpass filter with a first preset frequency band. Based on the fact that a respiratory rate range of the infant or the child is 20-40 bpm and a respiratory rate range of a normal 18-year-old adult is 10-30 bpm, compared to the adult, the child's respiration is fast and brief, and the frequency band of the bandpass filter used in the embodiments is adjusted to a relatively narrow range. Taking a bandpass filter with a frequency range of 0.2-0.6 HZ as an example, this embodiment may extract a more accurate respiratory signal of the child. The bandpass filter refers to a filter that passes frequency components in a certain frequency range but attenuates frequency components in other ranges to a very low level, such as an RLC oscillation loop.

The extracted respiratory signal is processed through mean filtering, and the respiratory rate is obtained based on the respiratory signal that has undergone the mean filtering.

It can be understood that various embodiments/implementations in the present disclosure may be combined with each other in case of no contradiction, which will not be elaborated herein.

What is claimed is:

1. A portable baby carrycot, comprising:
a carrycot body comprising a first side wall and a second side wall opposite to each other, and a support plate at a bottom, a lying area being above the support plate;
a support assembly configured to support the carrycot body, the support assembly comprising a first support part on the first side wall and a second support part on the second side wall;
a piezoelectric sensor arranged in the lying area to detect a respiratory movement of a baby;
a signal processing circuit configured to receive and process the respiratory movement detected;
a controller configured to generate a prompt message indicating a sleep state of the baby when the received and processed respiratory movement satisfies a preset condition; and
an alert device configured to provide a caregiver with the prompt message indicating the sleep state of the baby,
wherein a length of the piezoelectric sensor is greater than a width of the piezoelectric sensor, and a direction of a connection line between the first support part and the second support part is orthogonal to a direction of the length of the piezoelectric sensor.

2. The portable baby carrycot according to claim 1, wherein the support assembly further comprises a handle, and two ends of the handle are connected to the first support part and the second support part, respectively;
wherein a length direction of the handle is orthogonal to the direction of the length of the piezoelectric sensor.

3. The portable baby carrycot according to claim 1, wherein the support assembly further comprises a connection assembly for connection with a frame; the connection assembly comprises a first connection assembly on the first side wall and a second connection assembly on the second side wall; the first connection assembly and the second connection assembly are configured to engage with a first groove and a second groove of the frame, respectively;
wherein a direction of a connection line between the first connection assembly and the second connection assembly is orthogonal to the direction of the length of the piezoelectric sensor.

4. The portable baby carrycot according to claim 1, wherein the length of the piezoelectric sensor is equal to a length of the lying area.

5. The portable baby carrycot according to claim 1, wherein the length of the piezoelectric sensor is smaller than a length of the lying area, and at least part of a head and a chest of the baby lying in the lying area is located on the piezoelectric sensor.

6. The portable baby carrycot according to claim 1, wherein the piezoelectric sensor is a one-piece thin film piezoelectric sensor.

7. The portable baby carrycot according to claim 1, wherein a sleeping pad is laid on the support plate, and the piezoelectric sensor is arranged within or on the sleeping pad.

8. A baby stroller, comprising:
a frame comprising a first side and a second side opposite to each other, the first side and the second side having a first groove and a second groove, respectively; and
a baby carrycot, comprising:
a carrycot body comprising a first side wall and a second side wall opposite to each other, and a support plate at a bottom, a lying area being above the support plate;
a support assembly configured to support the carrycot body, the support assembly comprising a connection assembly for connection with the frame, the connection assembly comprising a first connection assembly on the first side wall and a second connection assembly on the second side wall, and the first connection assembly and the second connection assembly being configured to engage with the first groove and the second groove, respectively;
a piezoelectric sensor arranged in the lying area to detect a respiratory movement of a baby;
a signal processing circuit configured to receive and process the respiratory movement detected;
a controller configured to generate a prompt message indicating a sleep state of the baby when the received and processed respiratory movement satisfies a preset condition; and
an alert device configured to provide a caregiver with the prompt message indicating the sleep state of the baby;
wherein a length of the piezoelectric sensor is greater than a width of the piezoelectric sensor, and a direction of a connection line between the first connection assembly and the second connection assembly is orthogonal to the direction of the length of the piezoelectric sensor.

9. The baby stroller according to claim 8, wherein the support assembly further comprises a first support part on the first side wall and a second support part on the second side wall, and a direction of a connection line between the first support part and the second support part is orthogonal to a direction of the length of the piezoelectric sensor;
the support assembly further comprises a handle, and two ends of the handle are connected to the first support part and the second support part, respectively; a length direction of the handle is orthogonal to the direction of the length of the piezoelectric sensor.

10. The baby stroller according to claim 8, wherein the piezoelectric sensor is a one-piece thin film piezoelectric sensor.
